(19) United States
(12) Patent Application Publication
(10) Pub. No.: US 2020/0147165 A1
(43) Pub. Date: May 14, 2020

(54) COMPOSITION AND METHOD FOR PREPARING PAIN RELIEVING DRUG

(71) Applicant: Deyu SUN, El Monte, CA (US)

(72) Inventor: Deyu SUN, El Monte, CA (US)

(21) Appl. No.: 16/741,762

(22) Filed: Jan. 14, 2020

(51) Int. Cl. A61K 36/76; A61K 36/236; A61K 36/714; A61K 36/258; A61K 36/324; A61K 36/8884; A61K 36/48; A61K 35/655; A61K 9/16; A61K 9/48; A61K 35/02; A61K 36/73; A61K 31/125

(52) U.S. Cl. CPC A61K 36/76; A61K 2236/333; A61K 36/714; A61K 36/258; A61K 36/324; A61K 36/8884; A61K 36/48; A61K 35/655; A61K 9/16; A61K 9/48; A61K 35/02; A61K 36/73; A61K 31/125; A61K 2236/51; A61K 36/236

(57) ABSTRACT

The present invention discloses a composition for preparing a pain relieving drug, which includes poplar bark, Ligusticum wallichii, curcumin, ambergris, processed Radix Aconiti Lateralis, ray cartilage extract, wild ginseng, fructus psoraleae, garden balsam stem, pratt mountainash root-bark, annona muricata, Arisaema heterophyllum Blume, resina draconis, frankincense, myrrh, Brazilian sapodilla and camphor. The composition can be added with an appropriate auxiliary material to be made into dosage forms suitable for oral administration or external use, such as oral liquid, a patch, a liniment, a sustained-release agent and the like.

### BACKGROUND OF THE INVENTION

The present invention relates to the field of medicine, and in particular to a composition and method for preparing a pain relieving drug.

Epidemiological surveys show that more than 85% of adults suffer from various degrees of pain. At present, the number of patients suffering from pain is increasing year by year and the patients are getting younger. The annual economic loss amounts to more than 1.7 billion yuan. About more than 3% of the patients have been retired for illness or left their jobs due to severe pain in joints, waist and legs. According to incomplete statistics, more than 97% of the patients have died or are disabled due to rheumatoid diseases and gout. For rheumatoid diseases and gout, western medicine treatment has no specific drugs for treatment except for using hormones to diminish inflammation and relieve pain, and oral administration of western medicines has serious toxic and side effects, which seriously affects liver, kidney and the hematopoietic function.

At present, there is still no unified standard for the treatment of rheumatic arthralgia. With the continuous development of medical scientific-research technology, a traditional Chinese medicine preparation for external use with advantages of transdermal administration, strong permeability, quick response, a lasting effect, definite safe and reliable therapeutic effects, small toxic and side effects, strong pertinence, convenient use, wide drug sources with a low cost and easy availability has been developed, which opens up a new way for the treatment of advanced cancers, rheumatic pains and gout in the future.

### BRIEF SUMMARY OF THE INVENTION

An objective of the present invention is to provide a composition for preparing a pain relieving drug and a preparation method of the drug. The present invention is a drug prepared by the composition, which has the effects of dispelling wind and eliminating dampness, diminishing inflammation and relieving pain, relaxing tendons and activating collaterals, and particularly has a remarkable effect on the pain of an advanced cancer. The drug of the present invention is made into a patch and a liniment, and is a traditional Chinese medicine preparation for external use, with a good therapeutic effect and small side effects for treating pains of an advanced cancer, rheumatoid arthritis, rheumatic arthritis, a rheumatic heart disease, gout and complications caused by gout.

The composition for preparing the pain relieving drug consists of the following components in parts by weight: 50-100 parts of poplar bark, 15-20 parts of Ligusticum wallichii, 10-15 parts of curcumin, 0.1-0.3 parts of ambergris, 10-12 parts of processed Radix Aconiti Lateralis, 50-150 parts of ray cartilage extract, 35-50 parts of wild ginseng, 15-30 parts of fructus psoraleae, 30-70 parts of garden balsam stem, 25-30 parts of pratt mountainash root-bark, 100-200 parts of annona muricata, 50-100 parts of Arisaema heterophyllum Blume, 50-100 parts of resina draconis, 15-25 parts of frankincense, 15-25 parts of myrrh, 20-30 parts of Brazilian sapodilla, and 3-5 parts of camphor.

Preferably, the composition for preparing the pain relieving drug includes the following components in parts by weight: 80 parts of poplar bark, 18 parts of Ligusticum wallichii, 12 parts of curcumin, 0.2 parts of ambergris, 11 parts of processed Radix Aconiti Lateralis, 100 parts of ray cartilage extract, 40 parts of wild ginseng, 20 parts of fructus psoraleae, 50 parts of garden balsam stem, 28 parts of pratt mountainash root-bark, 150 parts of annona muricata, 80 parts of Arisaema heterophyllum Blume, 80 parts of resina draconis, 20 parts of frankincense, 20 parts of myrrh, 25 parts of Brazilian sapodilla, and 4 parts of camphor.

Preferably, the composition further includes 30% microcrystalline cellulose, 1% magnesium stearate, 3% sodium dodecyl sulfate, 60 grams of hydroxypropyl methylcellulose, an appropriate amount of 75% ethanol, 3 grams of propylene glycol, and 1 grams of polyethylene glycol 6,000.

This composition can be added with an appropriate auxiliary material to be made into dosage forms suitable for oral administration or external use, such as oral liquid, a patch, a liniment, a sustained-release agent and the like.

A method for preparing a pain relieving drug for external use includes the following preparation steps:

Step 1: weighing raw herbal materials according to a formula amount, taking Arisaema heterophyllum Blume, resina draconis, frankincense, myrrh at 90% of the formula amount, soaking them in 75% ethanol as a solvent for 48 hours, percolating at a speed of 1 ml/min per kilogram, collecting a filtrate, recovering the ethanol, and continually concentrating the filtrate to obtain a extractum with a relative density of 1-1.10;

Step 2: adding resina draconis, camphor and water, and adding the remaining 10% drugs of Arisaema heterophyllum Blume, curcumin, frankincense, myrrh and the like, grinding with a colloid mill to be ready for use, taking gelatin and adding water into it to dissolve it at 60° C., adding glycerol and diatomite into it, and uniformly mixing with the aforementioned ready-for-use solutions of Arisaema heterophyllum Blume and the like to prepare coating liquid; and

Step 3: coating, cutting and preparing the prepared coating liquid into a patch.

A method for preparing a pain relieving drug for external use includes the following preparation steps:

Step 1: weighing raw herbal materials according to a formula amount, taking a poplar bark extract, a processed Radix Aconiti Lateralis extract, fructus psoraleae raw material powder, an annona muricata extract, curcumin and the like raw material powder, microcrystalline cellulose and starch at the formula amount, placing all of them into a fluidized bed, according to parameters of an operation parameter list, spraying a proper amount of distilled water onto them for binding, stop spraying when pellets are grown to a certain size, appropriately drying the pellets, sieving, and selecting 30-mesh and 60-mesh pellets;

Step 2: placing the drug-loaded pellets into a fluidized bed, and according to parameters on an operation parameter list, spraying slow-release coating liquid onto the surface of the drug-loaded pellets for surface sealing, stop spraying after the coating weight is increased by 5%, and continually drying at 55-60° C. for 0.5 h to obtain film-coated sustained-release pellets; and

Step 3: encapsulating the 30-mesh pellets into capsules, and packaging the 60-mesh pellets to obtain traditional Chinese medicine of sustained-release capsules and pellets.

Nature flavor and channel tropism, and pharmacological actions:

(1) Poplar bark extract: bitter, sour, cold and nontoxic. Poplarpid has a wide application prospect in aspects of medicine. On Page 2037 of Compendium of Materia Medica, which is the first pharmacohistory book that records the medicinal efficacy and application range of poplar bark, it states that “it is used for (major function) poison wind and pedal edema, slow, weak and involuntary moving limbs, and is orally administrated after soaking in wine”, which indicates that the poplar bark has a certain therapeutic effect in treating rheumatalgia and skin diseases. (The inventor has studied and applied poplar bark for more than 50 years to treat rheumatic arthritis, rheumatoid arthritis and gout, traumatic injury, and pain caused by various advanced cancers, and has indeed achieved success. The inventor has helped tens of thousands of people to relieve the pain and to avoid the life-long injury of them due to amputation and paralysis).

Ligusticum wallichii extract: acrid in taste and warm in nature. It acts on liver, gallbladder and heart meridian channels. It has effects of activating blood circulation to dissipate stasis, relieving swelling and pain, promoting the circulation of qi, and relieving depression.

(3) Ambergris: bitter, sour, and neutral in nature. It acts on two meridian channels of liver and kidney. It has effects of removing blood stasis, dredging collaterals, nourishing liver and kidney, strengthening bones and muscles, promoting blood circulation, and relieving pain.

(4) Curcumin: bitter and pungent acrid in taste, and warm in nature. It acts on liver and spleen meridian channels. It has functions of relaxing meridian channels and relieving pain, promoting blood circulation and removing blood stasis, relieving rheumatic osteodynia, removing wind and dampness, dispelling cold and relieving swelling and pain, and has therapeutic effects on for example all of chest distress and heartache, a coronary heart disease and angina pectoris.

(5) Fructus psoraleae extract: bitter in taste, and warm in nature. It acts on liver and kidney meridian channels. It has effects of tonifying kidney, enriching essence, strengthening the bones, relieving pain after injury, also inhibiting damage to the liver caused by many western medicines, and increasing detoxification and metabolic functions of the liver. The five drugs are all sovereign drugs.

(6) Processed Radix Aconiti Lateralis extract: acrid and sweet in taste, extremely hot in nature, toxic for raw use, and only prepared into a drug after being steamed for nine times and sun-dried for several times. It acts on heart, spleen and kidney meridian channels. Efficacy: it is a strong medicine with functions of rescuing from collapse by restoring yang, tonifying fire and helping yang, dispelling cold and relieving pain, dispelling cold phlegm, and easing joint movement.

(7) Arisaema heterophyllum Blume: slightly bitter in taste, and warm in nature. It acts on liver and stomach meridian channels. Efficacy: it is a good medicine for removing blood stasis, stopping bleeding, promoting blood circulation and relieving pain.

(8) Garden balsam stem: sweet and acrid in taste, and warm in nature. It acts on lung, liver and kidney channels. Efficacy: dispelling wind and dampness, relaxing tendons and activating collaterals, dispelling cold and relieving swelling, detoxifying and relieving pain.

(9) Camphor: acrid and bitter in taste, and slightly cold. It acts on heart, spleen and lung meridian channels. Efficacy: inducing resuscitation, clearing heat and relieving pain.

(10) Annona muricata: sweet in taste, and warm in nature. It acts on spleen, stomach, liver and kidney meridian channels. Efficacy: preventing diseases, resisting cancers, and relieving swelling and pain. The five drugs are all ministerial drugs.

(11) Pratt mountainash root-bark: acrid in taste, and hot in nature. It acts on spleen, stomach and kidney meridian channels. Efficacy: warming the middle warmer, dispelling cold, relieving pain, eliminating dampness, killing parasites and relieving itching.

(12) Resina draconis: neutral in nature, and sweet and salty in taste. It acts on heart and liver meridian channels. Efficacy: dissipating blood stasis and relieving pain, stopping bleeding and promoting granulation, promoting blood circulation and removing blood stasis, relieving swelling and dissipating stagnation, etc.

(13) Frankincense and myrrh: bitter in taste, and neutral in nature. It acts on heart, liver and spleen meridian channels. Efficacy: promoting blood circulation and relieving pain, relieving swelling, promoting granulation and growing flesh. The above four drugs are all assistant drugs.

(14) The ray cartilage extract has rich nutritional value, contains special components that can enhance human immunity and memory, contains calcium and trace elements needed by human body, and is easy to be absorbed by the human body.

(15) Brazilian sapodilla: sweet and slightly bitter in taste, warm and neutral in nature. It acts on heart, spleen and lung meridian channels. Efficacy: powerfully enriching pri-mordial qi, relieving desertion, engendering liquid and allaying thirst, calming the nerves and improving intelligence.

Additionally, the compatibility of the various drugs has a better therapeutic effect.

Beneficial effects of the present invention:

The drug prepared from the composition can promote the hematopoietic function of bone marrow cells, has the effects of generating essence and supplementing qi and blood, is suitable for people with deficiency of both qi and blood caused by various reasons, and is especially suitable for tumor patients with hematopoietic cell dysfunction caused by radiotherapy and chemotherapy. It can also be used for adjuvant treatment of various anemia and immune system diseases. It has functions of dispelling wind, dispelling cold, removing dampness, activating collaterals, relieving pain, relieving swelling and dissipating stagnation, and improving internal and external pains of the body, and has therapeutic effects on advanced cancers, gout, rheumatic arthritis, angina pectoris, etc.

### DETAILED DESCRIPTION OF THE INVENTION

The present invention will be further described below with reference to specific examples:

### Example 1: Preparing a Composition into Oral Liquid

50-100 parts of poplar bark, 15-20 parts of Ligusticum wallichii, 10-15 parts of curcumin, 0.1-0.3 parts of ambergris, 10-12 parts of processed Radix Aconiti Lateralis, 50-150 parts of ray cartilage extract, 35-50 parts of wild ginseng, 15-30 parts of fructus psoraleae, 30-70 parts of garden balsam stem, 25-30 parts of pratt mountainash root-bark, 100-200 parts of annona muricata, 50-100 parts of Arisaema heterophyllum Blume, 50-100 parts of resina draconis, 15-25 parts of frankincense, 15-25 parts of myrrh, 20-30 parts of Brazilian sapodilla, and 3-5 parts of camphor were prepared into oral liquid.

The preparation method is as follows: (1) the poplar bark, Ligusticum wallichii, curcumin, ambergris, processed Radix Aconiti Lateralis, ray cartilage extract, wild ginseng, fructus psoraleae, garden balsam stem, pratt mountainash root-bark, annona muricata, Arisaema heterophyllum Blume, resina draconis, frankincense, myrrh, Brazilian sapodilla, and camphor are crushed.

(2) the raw material powder was weighed at the aforementioned weight, added with distilled water under stirring, and extracted at a controlled temperature of 80° C. for 1 hour, and filtered, the filter residue was added with water, extracted at 80° C. for 0.5 h, and filtered, and the filtrates were combined and concentrated at 80° C. under reduced pressure.

(3) Formulating: the above concentrated solutions were uniformly mixed, added with maltooligosaccharide and glucose, filtered, sterilized, and encapsulated to obtain the product.

### Example 2: Preparing a Composition into a Patch, a Liniment

The poplar bark, Ligusticum wallichii, curcumin, ambergris, processed Radix Aconiti Lateralis, ray cartilage extract, wild ginseng, fructus psoraleae, garden balsam stem, pratt mountainash root-bark, annona muricata, Arisaema heterophyllum Blume, frankincense, myrrh, and Brazilian sapodilla were taken at 90% of the formula amount, soaked in 75% ethanol as a solvent for 48 hours, percolated at a speed of 1 ml/min per kilogram, a filtrate was collected, the ethanol was recovered, and the filtrate was continually concentrated to obtain a extractum with a relative density of 1.08-1.10. The extractum was added with resina draconis, camphor and water, and added with the remaining 10% drugs of poplar bark, Ligusticum wallichii, curcumin, ambergris and the like, grinded with a colloid mill to be ready for use, gelatin was taken and dissolved at 60° C. by adding water into it, added with glycerol and diatomite, and uniformly mixed with the aforementioned ready-for-use solutions of Arisaema heterophyllum Blume and the like to prepare coating liquid, coated and cut to obtain the patch.

This product adopted a water-soluble transdermal matrix, was externally applied onto a cancer pain part for percutaneous absorption, which was beneficial for the drug to exert its functions of effectively relieving pain and swelling and could effectively improves the pain for a patient with an advanced cancer.

The preparation method of the liniment: the aforementioned raw herbal materials were weighed in proportion according to proportion parameters, added with 9 times amount of water and decocted for 2-3 hours, extracted for 3 times, and filtered. The filter residue was added with another 3 times amount of water and decocted for 2 hours, and filtered. The filtrates were combined, and concentrated under reduced pressure to a thick paste with a relative density of 1.13-1.17 (85° C.). The thick paste was added with ethanol to make the ethanol content of the drug solution reach 75%, added with polyvinyl formal-acetal, completely dissolved after added with 75% ethanol for soaking and expanding, mixed well, and then ready for use. The alcohol deposit fluid was added with the pratt mountainash root-bark extract, fully stirred with the matrix to uniform, added with 75% ethanol to adjust the total amount to 1,000 grams, and packaged to obtain the liniment.

This product adopted a water-soluble transdermal matrix, was externally applied onto a cancer pain part for percutaneous absorption, which was beneficial for the drug to exert its functions of effectively relieving pain and swelling and could effectively improves the pain for a patient with an advanced cancer.

### Example 3: Preparing the Composition into a Sustained-Release Preparation

The above poplar bark, processed Radix Aconiti Lateralis, fructus psoraleae, annona muricata, curcumin and the like raw material extract powder, microcrystalline cellulose and starch were all placed in a fluidized bed, sprayed with a proper amount of distilled water according to parameters in an operation parameter list, spraying was stopped when the pellets were controlled at about 0.18-0.22 grams, and appropriate drying was conducted. Sieving was conducted to select pellets between 30 meshes and 60 meshes;

(3) The drug-loaded pellets were placed into a fluidized bed, and according to parameters on an operation parameter list, slow-release coating liquid was sprayed onto the surface of the drug-loaded pellets for surface sealing, Spraying was stopped after the coating weight was increased by 5%. Drying was continually done at 55-60° C. for 0.5 h to obtain film-coated sustained-release pellets. The 30-mesh pellets were encapsulated into capsules, and the 60-mesh pellets were packaged to obtain traditional Chinese medicines of sustained-release capsules and pellets.

Characteristics: the sustained-release technology for preparing this product could effectively control the drug to be released within a certain period of time, maintain an effective blood drug concentration for 24 hours, has no burst release phenomenon, and has no irritation and adverse reactions to stomach, intestine, liver and spleen.

Observation of therapeutic effects:

Referring to the liniment of Example 2, the liniment was respectively applied to the first group and the second group. The liniment was applied to the pain part 2-3 times a day (for a patient with a severe pain, administration for 5-6 times was allowed within 24 hours). 20 days was one course of treatment. After two courses of treatment, the pains of all patients were obviously alleviated, without any adverse reaction found in the medication process. In six years, 42 patients had improved walking, no longer suffered from pain, and had no relapse reaction after drug withdrawal.

In 10 years, of 8 patients, 7 were basically cured after 4 courses of treatment, and 1 had other symptoms obviously alleviated except a leg pain due to prolapse of lumbar intervertebral disc. Summary of effects: a significant effect of 85%, an effective rate of 98%, and no obvious effect of 3% were achieved. It could be seen from the examples that this traditional Chinese medicine formula had the advantages of quick effect, low cost, safety and reliability, small toxic and side effects and the like.

Observation of recipe cases:

It was used in 46 cases of advanced-cancer pains (statistics of some outpatients). The results showed a marked effect (pain disappeared) in 37 cases, improvement (pain relief) in 19 cases, ineffectiveness in 2 cases, with a total effective rate of 98%.

A typical case was Mrs. Lu, who was 56 years old and went to see a doctor at 9:00 p.m. on Mar. 5, 2018. She complained that she had a severe abdominal pain in the past two days, was unable to eat or drink, and the pain was even more severe at night. She had gone to two tumor hospitals for examination and was diagnosed as massive hepatocellular carcinoma and was not given treatment. Current conditions: the patient had a face that was as yellow as paper, had thin muscle, was skinny, had yellow skin and sclera, and was extremely poor in spirit. Auscultation: the patient had normal heart and lung, a large liver below umbilicus with a harder texture and a nodular shape. Upon abdominal CT, the patient was reported to have a massive hepatocellular carcinoma. According to the diagnosis of the pulse condition of the patient, the six pulses were deep and stagnating, the tongue was light-colored with a white coating that was gray and had ecchymosis. The patient was thus diagnosed as having Qi-stagnation and blood stasis, phlegm accumulation and damp obstruction. Seeing the patient's unbearable pain, she was given an external patch (Example 2) and pellets for oral administration (Example 3), and the pain began to decrease in about 35 minutes. The patch was replaced every 3-5 days. The pellets were administrated 1-2 times a day, with 5-7 pellets each time. The treatment was conducted for 58 days in total. Then the liver was reduced from 5 cm below the navel to 7 cm above the navel. The patient had a normal diet. The body weight of the patient was 53 kilograms before the treatment, and increased to 69 kilograms after the 58 days of treatment. The patient could not move before the treatment, and could walk in the park and drive for 1 hour without feeling tired after the treatment. The tumor-bearing patient had survived for more than 5 years, and was still healthy without relapse after follow-up.

What is claimed is:

1. A composition for preparing a pain relieving drug, wherein the composition comprises the following components in parts by weight: 50-100 parts of poplar bark, 15-20 parts of Ligusticum wallichii, 10-15 parts of curcumin, 0.1-0.3 parts of ambergris, 10-12 parts of processed Radix Aconiti Lateralis, 50-150 parts of ray cartilage extract, 35-50 parts of wild ginseng, 15-30 parts of fructus psoraleae, 30-70 parts of garden balsam stem, 25-30 parts of pratt mountainash root-bark, 100-200 parts of annona muricata, 50-100 parts of Arisaema heterophyllum Blume, 50-100 parts of resina draconis, 15-25 parts of frankincense, 15-25 parts of myrrh, 20-30 parts of Brazilian sapodilla, and 3-5 parts of camphor.

2. The composition for preparing a pain relieving drug according to claim 1, wherein the composition comprises the following components in parts by weight: 80 parts of poplar bark, 18 parts of Ligusticum wallichii, 12 parts of curcumin, 0.2 parts of ambergris, 11 parts of processed Radix Aconiti Lateralis, 100 parts of ray cartilage extract, 40 parts of wild ginseng, 20 parts of fructus psoraleae, 50 parts of garden balsam stem, 28 parts of pratt mountainash root-bark, 150 parts of annona muricata, 80 parts of Arisaema heterophyllum Blume, 80 parts of resina draconis, 20 parts of frankincense, 20 parts of myrrh, 25 parts of Brazilian sapodilla, and 4 parts of camphor.

3. A method for preparing a pain relieving drug for external use, comprising the following preparation steps:
Step 1: weighing raw herbal materials according to a formula amount, taking Arisaema heterophyllum Blume, resina draconis, frankincense, myrrh at 90% of the formula amount, soaking them in 75% ethanol as a solvent for 48 hours, percolating at a speed of 1 ml/min per kilogram, collecting a filtrate, recovering the ethanol, and continually concentrating the filtrate to obtain a extractum with a relative density of 1.08-1.10;
Step 2: adding 5 grams of camphor and 25 milliliters of distilled water, and adding the remaining 10% drugs of Arisaema heterophyllum Blume, curcumin, frankincense, myrrh and the like, grinding with a colloid mill to be ready for use, taking gelatin and adding water into it to dissolve it at 60° C., adding glycerol and diatomite into it, and uniformly mixing with the aforementioned ready-for-use solutions of Arisaema heterophyllum Blume and the like to prepare coating liquid; and
Step 3: coating, cutting and preparing the prepared coating liquid into a patch.

4. A method for preparing a pain relieving drug for external use, comprising the following preparation steps:
Step 1: weighing raw herbal materials according to a formula amount, taking poplar bark, processed Radix Aconiti Lateralis, fructus psoraleae raw material powder, annona muricata, curcumin and the like raw material powder, microcrystalline cellulose and starch at the formula amount, placing all of them into a fluidized bed, spraying a proper amount of distilled water onto them for binding, stop spraying when pellets are controlled to be about 0.18-0.22 grams, and drying the pellets at 60° C. for 2 hours, sieving to select 30-mesh and 60-mesh pellets;
Step 2: placing the drug-loaded pellets into a fluidized bed, and spraying slow-release coating liquid onto the surface of the pellets for surface sealing, stop spraying after the coating weight is increased by 5%, continually drying at 55-60° C. for 0.5 h to obtain film-coated sustained-release pellets; and
Step 3: encapsulating the 30-mesh pellets into capsules, and packaging the 60-mesh pellets to obtain traditional Chinese medicine of sustained-release capsules and pellets.
